(19) United States
(12) Patent Application Publication
(10) Pub. No.: US 2011/0039888 A1
(43) Pub. Date: Feb. 17, 2011

(54) ASYMMETRIC ALPHA FUNCTIONALIZATION AND ALPHA, ALPHA BISFUNCTIONALIZATION OF ALDEHYDES AND KETONES

(76) Inventors: Don M. Coltart, Durham, NC (US); Daniel Lim, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/736,580

(22) PCT Filed: Apr. 21, 2009
(86) PCT No.: PCT/US2009/002450

§ 371 Date: Oct. 20, 2010

Related U.S. Application Data
(62) Division of application No. 61/071,279, filed on Apr. 21, 2008.

(51) Int. Cl. A61K 31/4709; C07D 413/12; C07D 215/14; C07D 401/06; C07D 263/28; C07D 211/32; A61K 31/451; A61K 31/4178; C07C 45/00; A61P 33/06; A61P 25/28; A61P 1/08

(52) U.S. Cl. 514/314; 546/175; 546/174; 546/205; 548/230; 546/206; 548/311.4; 514/319; 514/397; 568/388; 568/351

(57) ABSTRACT

The present invention relates, generally, to asymmetric α-functionalization and to asymmetric α,α-bisfunctionalization of ketones and aldehydes and, in particular, to chiral auxiliaries suitable for use in effecting such functionalizations and to methods of using same.

The present application claims priority from U.S. Provisional Application 61/071,279, filed Apr. 21, 2008, the entire content of which is incorporated herein by reference.

### TECHNICAL FIELD

The present invention relates, generally, to asymmetric α-functionalization and to asymmetric α,α-bisfunctionalization of ketones and aldehydes and, in particular, to chiral auxiliaries suitable for use in effecting such functionalizations and to methods of using same.

### BACKGROUND

Carbon-carbon and carbon-heteroatom bond-forming reactions are among the most important synthetic transformations practiced in the modern pharmaceutical industry and in academic and government laboratories throughout the world. These reactions have significant and wide-reaching implications in many areas of science but are most notable in the development and synthesis of new and existing pharmaceutical products. The ability to conduct such transformations in an asymmetric fashion is more critical than ever as the shift toward the development of chiral, non-racemic drugs increases.

Carbon-carbon and carbon-heteroatom bond-forming reactions can be categorized using simplified mechanistic criteria as either polar reactions, free-radical reactions, pericyclic reactions or transition metal-mediated reactions. Polar reactions are, by far, the most pervasive and have provided the foundation for the advancement of synthetic organic chemistry to its present state. The most useful approach to carbon-carbon and carbon-heteroatom bond formation via polar intermediates is the reaction of an enolate with an electrophile (FIG. 1). Enolate chemistry has been the subject of extensive study over the past several decades and is now considered an indispensable method for carbon-carbon and carbon-heteroatom bond formation.

Ketone or aldehyde α-functionalization via electrophilic addition to derived enolates is one of the most important and relied on classes of transformations in synthetic organic chemistry. Despite this, there is a scarcity of methods available for conducting such transformations asymmetrically. In the case of ketone and aldehyde α-functionalization in general, the use of derived azaenolates has proven more effective in terms of reactivity, product yield and selectivity, compared to enolates themselves. This also provides a means for incorporation of a nitrogen-based chiral auxiliary leading to asymmetric functionalization. The first asymmetric synthesis via an azacarbonyl system was reported by Yamada in 1969 (Yamada et al, Tetrahedron Lett. 48:4233-4236 (1969)). In this transformation, an enamine served as the nucleophilic component and was formed via condensation of the parent ketone and a proline-derived chiral auxiliary (FIG. 2A). While noteworthy as a pioneering approach to asymmetric ketone α-alkylation, the stereoselectivity obtained was moderate. In 1976, Koga and Myers independently reported the use of acyclic amino acid-derived auxiliaries in the asymmetric α-alkylation of ketones via derived imines (FIGS. 2B and 2C) (Hashimoto et al, Tetrahedron Lett. 6:573-576 (1978), Meyers et al, J. Am. Chem. Soc. 98:3032-3033 (1976), Meyers et al, J. Org. Chem. 43:3245-3247 (1978)). LDA-mediated metallation of the imines, followed by alkylation gave good to very good stereoselectivity in the case of cycloalkylones. Unfortunately, alkylation of acyclic ketones did not proceed with suitably high stereoselectivity. A further limitation of each of the above methods is that the enamine and imine intermediates are difficult to form quantitatively and are hydrolytically unstable (Yamada et al, Tetrahedron Lett. 48:4233-4236 (1969); Hashimoto et al, Tetrahedron. Lett. 6:573-576 (1978), Meyers et al, J. Am. Chem. Soc. 98:3032-3033 (1976), Meyers et al, J. Org. Chem. 43:3245-3247 (1978)). In contrast, hydrazones derived from the well-know SAMP and RAMP dialkyl hydrazine auxiliaries are stable and give good to excellent stereoselectivity and yield (FIG. 2D). Over the years, alkylation via SAMP/RAMP-derived hydrazones has evolved to become the current state of the art for asymmetric α-functionalization of aldehydes and ketones (see Enders D., Alkylation of Chiral Hydrazones, in Asymmetric Synthesis, 1st ed., Morrison, J. D., Ed. Academic Press, New York, 1984, Vol. 3, pp 275-339; Job et al, Tetrahedron 58(12):2253-2329 (2002)).

Since the introduction of the SAMP/RAMP hydrazone technology, considerable effort has been invested in studying and further developing these chiral ketone enolate equivalents. By the early part of this decade, it had been demonstrated that they react with synthetically useful yields and selectivities with a variety of electrophiles. Consequently, SAMP and RAMP have become the gold standard in terms of asymmetric ketone and aldehyde functionalization and have been used in numerous total syntheses.

While these auxiliaries (SAMP and RAMP) have proven extremely beneficial to synthetic chemistry, they have several important drawbacks that complicate their use from a practical perspective in small-scale applications, and preclude the possibility of large-scale applications in the context of, for example, drug manufacture. For instance, they are based on a proline core, which limits structural variation for improvement of selectivity, reactivity, etc. Furthermore, since their synthesis is non-trivial, they are expensive. These auxiliaries are liquids, and the derived hydrazones generally are as well, making them somewhat difficult to work with in comparison to solids. In addition, formation of the corresponding hydrazones requires long periods of reflux under dehydrating conditions. Due to the weakly acidic nature of dialkyl hydrazones, azaenolate formation is also a lengthy process requiring exposure, for example, to lithium diisopropylamide (LDA) for 2-10 h. Making matters especially difficult is the fact that, once the metallated species has finally been formed, it must be cooled to between −110° C. and −78° C. prior to functionalization for maximal stereoselectivity. Furthermore, conditions for auxiliary removal are often problematic (Enders et al, Accounts Chem. Res. 33(3):157-169 (2000)). Some hydrolytic methods are available but they are not general and/or require harshly acidic conditions, which can cause detrimental side reactions. As such, the most common methods for removal of ketone-derived dialkyl hydrazone auxiliaries rely on oxidation of the hydrazone double bond, or an amine quaternization/hydrolysis sequence. However, this liberates the auxiliary in an altered form that either requires additional synthetic transformations for recycling or makes recycling impossible, adding considerable cost and time to the asymmetric functionalization. Additionally, these methods are limited to compounds not having oxidatively-sensitive or nucleophilic functionality elsewhere in the molecule.

The present invention provides a new method for the asymmetric α-functionalization, and for the asymmetric α,α-bisfunctionalization, of ketones and aldehydes using chiral auxiliaries. These auxiliaries are simple to make and to use and overcome the practical limitations associated with the SAMP/RAMP auxiliaries. Further, they maintain extremely high levels of asymmetric induction.

### SUMMARY OF THE INVENTION

The present invention relates, in general, to asymmetric α-functionalization and asymmetric α,α-bisfunctionalization of ketones and aldehydes. More specifically, the invention relates to methods of effecting asymmetric α-functionalization and asymmetric α,α-bisfunctionalization and to chiral auxiliaries suitable for use in such methods. The instant method has application in many areas of synthetic chemistry and makes possible a means for conducting asymmetric ketone/aldehyde α-functionalization, or α,α-bisfunctionalization, on the scale required of, for instance, drug manufacturing processes.

Objects and advantages of the present invention will be clear from the description that follows.

### BRIEF DESCRIPTION OF THE DRAWINGS

FIG. 1. Enolate-mediated carbon-carbon bond formation.

FIGS. 2A-2D. Early examples of asymmetric ketone α-functionalization. FIG. 2A. Method of Yamada et al (Tetrahedron Lett. 48:4233-4236 (1969)). FIGS. 2B and 2C. Methods of Koga (Tetrahedron Lett. 6:573-576 (1978)) and Meyers et al (Am. Chem. Soc. 98 (10):3032-3033 (1976); J. Org. Chem. 43(16):3245-3247 (1978)). FIG. 2D. Hydrazones derived from SAMP and RAMP dialkyl hydrazine auxiliaries.

FIG. 3. General representation of the asymmetric ketone α-functionalization method of the invention.

FIG. 4. Activated hydrazones and N-amino cyclic carbamates.

FIG. 5. Asymmetric α,α-bisalkylation.

FIG. 6. Crystal structures.

FIG. 7. Stereochemistry of azaenolate formation and alkylation. L=large substituent, S=small substituent.

FIG. 8. Asymmetric α-alkylation and α,α-bisalkylation of ketones via chiral N-amino cyclic carbamates (ACCs).

FIGS. 9A-9C. Preparation of single enantiomer drugs. FIG. 9A. mefloquine, FIG. 9B. donepezil and FIG. 9C. ondanestron

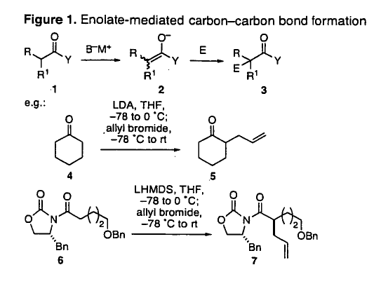

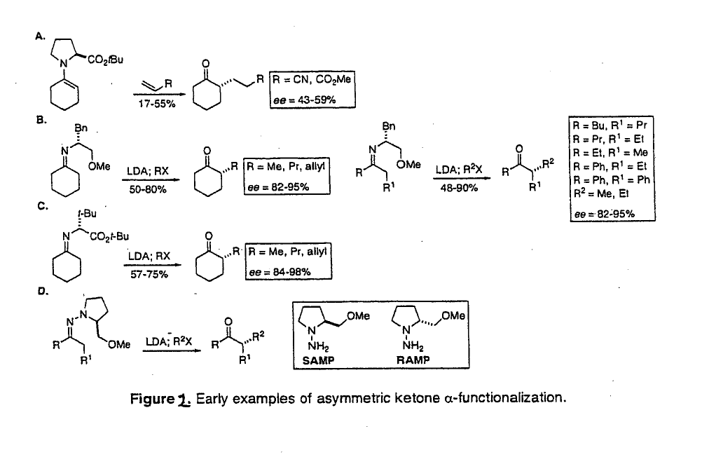

### DETAILED DESCRIPTION OF THE INVENTION

The present invention relates to a new class of chiral auxiliaries for the asymmetric α-functionalization, and α,α-bisfunctionalization, of ketones and aldehydes (e.g., the asymmetric α-(or α,α-bis) alkylation, amination, halogenation, oxidation, or thiolation). These auxiliaries are operationally simple to use, maintain extremely high levels of asymmetric induction and overcome the practical limitations associated with the SAMP/RAMP auxiliaries. The instant auxiliaries are easily prepared from naturally occurring building blocks and offer a degree of structural flexibility greater than that of the SAMP/RAMP auxiliaries. They allow straightforward structural tuning during optimization of selectivity, etc. The incorporation of these auxiliaries is a rapid and facile process and can be conducted at room temperature. Deprotonation of the auxiliary-derivatized substrates is rapid over a wide range of temperatures. Functionalization (e.g., alkylation) can be conducted at, for example, −40° C. to 0° C., which makes larger-scale asymmetric functionalizations possible. Following functionalization, the auxiliaries can be removed under mild conditions in an unaltered form and recovered quantitatively.

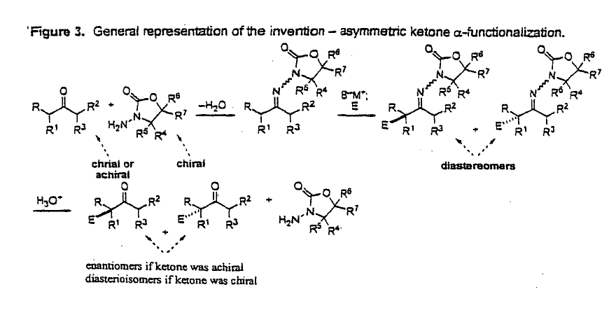

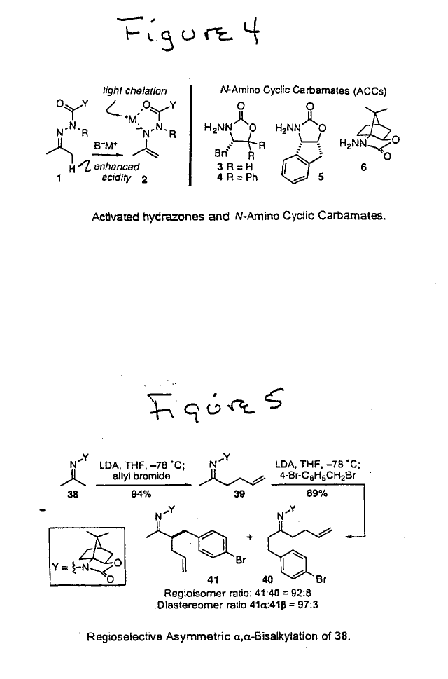

In accordance with one aspect of the invention, asymmetric α-functionalization of ketones and aldehydes is effected using a substituted hydrazide of the formula H₂N—C (wherein C is defined as indicated below). Examples of substituted hydrazides (auxiliaries) suitable for use in the invention include chiral N-amino cyclic carbamates (ACCs) and chiral N-amino cyclic sulfamates (ACSs). A preferred embodiment of the instant method of effecting α-functionalization of a ketone is shown in FIG. 3. A first step of this embodiment results in the formation of an activated hydrazone:

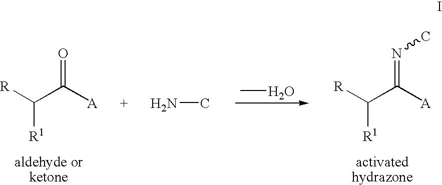

wherein

A=H or alkyl or substituted alkyl, etc. (e.g., A includes an alkyl or substituted alkyl having at least one hydrogen a to the carbonyl of the starting aldehyde or ketone),

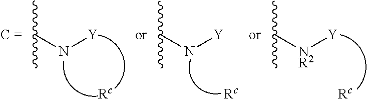

Y=electron withdrawing group such as CO, CO₂, SO, SO₂, CS, CR³N, (or, in the case of the second of the definitions of C above, COR⁴, CO₂R⁴, SOR⁴, SO₂R⁴, CSR⁴, CR³NR⁴), etc.;

R^{c}=chiral group

R, R¹, R², R³ and R⁴ are, independently, substituents, such as H, alkyl (linear, branched or cyclic), substituted alkyl (linear, branched or cyclic), aryl, substituted aryl, heteroatom (e.g., N, O, S, P, Se, etc.), halogen, Si, or B, wherein when C includes R² and R³ or R³ and R⁴, those R², R³ and R⁴ groups can be the same or different.

A further step results in the formation of a substituted activated hydrazone:

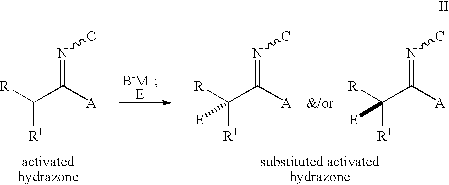

wherein B⁻M⁺ is any strong base capable of deprotonating the activated hydrazone (e.g., LDA, lithium hexamethyldisilazide (LHMDS), lithium isopropylcyclohexylamide (LICA), potassium hexamethyldisilazane (KHMDS), sodium hexamethyl disilazide (NHMDS), bromomagnesium diisopropylamide (BMDA), n-butyllithium (n-BuLi), sec-BuLi, t-BuLi, sodium hydride (NaH) or lithium hydride (LiH)),

wherein E is any electrophile capable of reacting with an enolate or azaenolate (e.g., an alkyl halide, alkyl sulfonate, α-β-unsaturated aldehyde, α-β-unsaturated ketone, α-β-unsaturated ester, α-β-unsaturated thioester, α-β-unsaturated amide, α-β-unsaturated sulfonate, nitro olefin, aldehydes, ketone, acid halide, ester, thioester, acylating agent, epoxide, aziridine, imine, N-substituted imine, electrophilic oxygen species, electrophilic nitrogen species, electrophilic sulfur species, halogen, electrophilic silicon species or electrophilic boron species), and

wherein A, C, Y, R^{c}, R, R¹, R², R³ and R⁴ are as defined above.

Another step yields the α-functionalized aldehyde or ketone and the auxiliary:

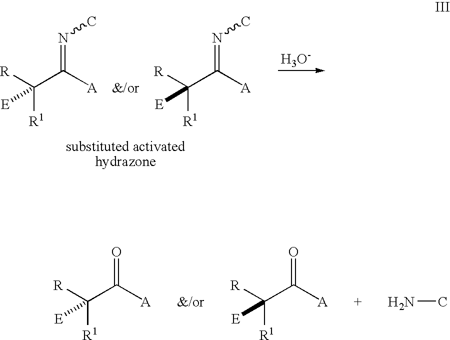

Auxiliaries suitable for use in the invention (e.g., ACCs and ACSs) can be prepared, for example, from naturally occurring building blocks, such as amino acids. (See Example that follows.) A preferred auxiliary is of the formula:

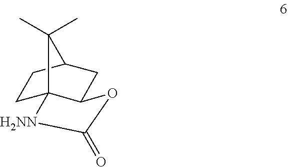

As indicated above, the present invention comprises, as a first step, the reaction of a chiral auxiliary of the invention with a ketone or aldehyde to form an activated hydrazone (e.g., step I above). This reaction can be carried out, for example, at a temperature between 25° and 110° C., for example, in an organic solvent such as methylene chloride, benzene, or toluene. Preferably, the reaction is conducted at room temperature in the presence of a mild acid, such as p-TsOH.

The electron withdrawing substituent (e.g., see Y definition above) of the activated hydrazones of the invention renders them highly acidic. Thus, deprotonation (e.g., step II above) is rapid over a wide range of temperatures (e.g., −110° C. to 0° C., preferably, −60° C. to 0° C.). Furthermore, the relatively high electron density on the electron withdrawing substituent in the derived azaenolates provides stronger metal cation chelation. This can be leveraged to enable high selectivity in the alkylation even at temperatures in the range of, for example, −60° C. to 0° C., thereby making large-scale applications possible.

Substituted activated hydrazones of the invention can be cleaved under mild conditions without damaging the parent auxiliary (see, for example, step III above). Suitable reaction conditions include those described in the Example that follows. Following hydrolysis of the hydrazone, the auxiliary can be recovered by extraction into a suitable organic solvent using standard work up procedures and purified as necessary via crystallization or chromatography.

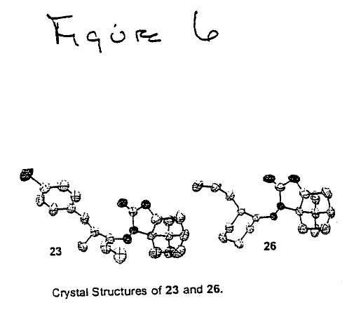

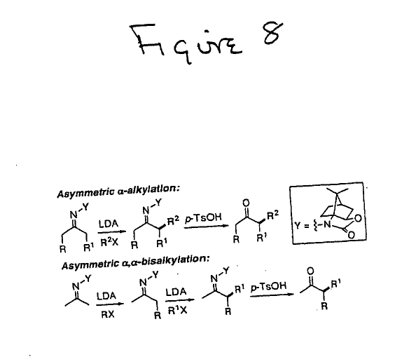

In a further aspect of the invention, asymmetric α,α-bisfunctionalization of a ketone or aldehyde is effected using a substituted hydrazide of formula H₂N—C (auxiliary), as described above. A first step of this aspect corresponds essentially to step I above. In a further step, deprotonation and then addition of a first electrophile proceeds as described in step II above with the formation of a substituted activated hydrazone. In a third step, the substituted activated hydrazone resulting from step II is subjected to further deprotonation and then addition of a second electrophile to yield a di-substituted activated hydrazone which can be cleaved as described in step III above to yield the α,α-bisfunctionalized ketone or aldehyde and the auxiliary. (See, for example, FIGS. 5 and 8).

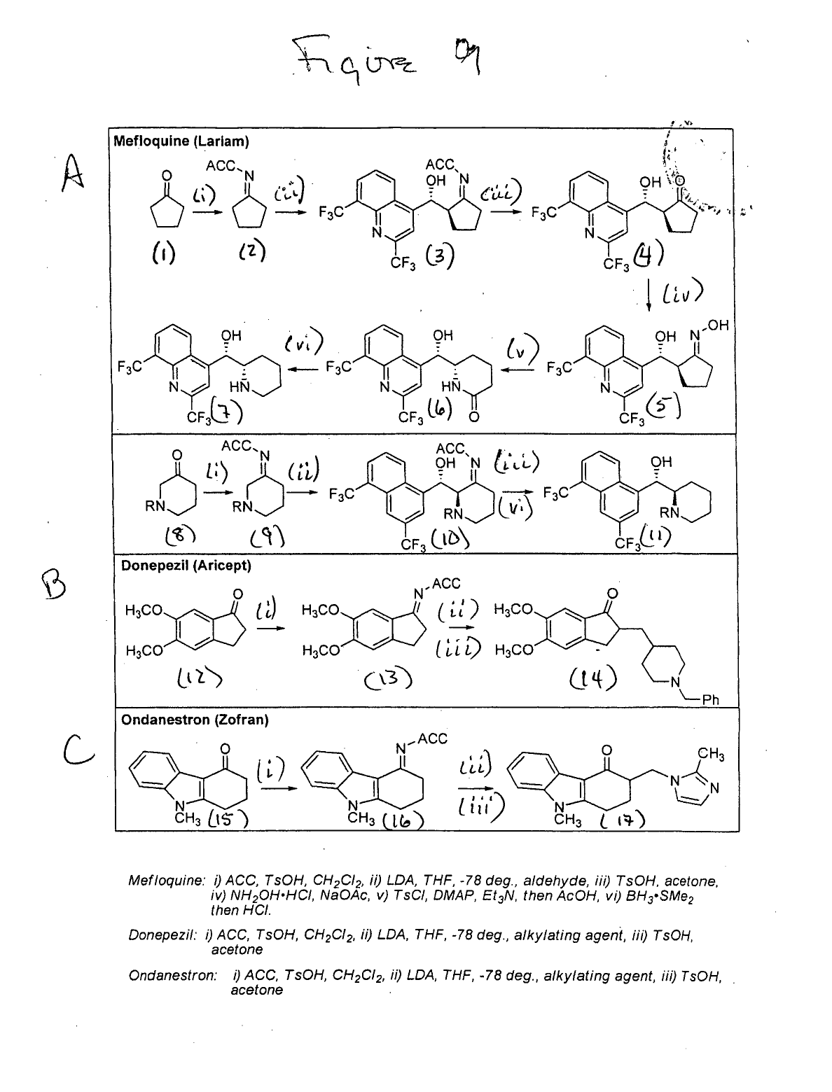

As pointed out above, the method of the instant invention has application in many areas of synthetic chemistry, including drug manufacturing. Specific examples of the preparation of single enantiomer drugs (i.e., mefloquine, donepezil and ondanestron) are described in Example 2 and depicted in FIG. 9. The invention includes the methods of synthesis described as well as the novel intermediates and products. In that regard, the present invention relates to compounds (4), (5), (6) and (7) as shown in FIG. 9A, both as racemic mixtures and as single enantiomers. That is, the invention relates to the (+)-enantiomer of, for example, compound (7) free of the (−)-enantiomer, as well as to the (−)-enantiomer free of the (+)-enantiomer, likewise for compounds (4)-(6). The invention further relates to the (+)-enantiomer of compound (11) (e.g., when R is hydrogen) free of the (−)-enantiomer, as well as to the (−)-enantiomer of compound (11) free of the (+)-enantiomer. The invention additionally relates to the (+)-enantiomers of compounds (14) and (17) of FIGS. 9B and 9C, respectively, free of the (−)-enantiomers, as well as to the (−)-enantiomers of compounds (14) and (17) free of the (+)-enantiomers. The invention also relates to (−)- and (+)-enantiomers of derivatives of the compounds depicted in FIG. 9, for example, derivatives of compounds (4)-(7), (11), (13), (14), (16), and (17).

In addition to the methods and compounds described in Example 2 and shown in FIG. 9, the invention also includes compositions comprising such compounds (or, as appropriate, pharmaceutically acceptable salts thereof) and a carrier, e.g., a pharmaceutically acceptable carrier.

Referring to the compounds depicted in FIG. 9, the invention further includes a method of treating or preventing malaria comprising administering to a patient in need thereof an amount of the (+)-enantiomer of compound (11—e.g., when R is hydrogen), or pharmaceutically acceptable salt thereof, free of the (−)-enantiomer of compound (11—e.g., when R is hydrogen), or pharmaceutically acceptable salt thereof, sufficient to effect the treatment or prevention. Alternatively, the invention includes a method of treating or preventing malaria comprising administering to a patient in need thereof an amount of the (−)-enantiomer of compound (11—e.g., when R is hydrogen), or pharmaceutically acceptable salt thereof, free of the (+)-enantiomer of compound (11—e.g., when R is hydrogen), or pharmaceutically acceptable salt thereof, sufficient to effect the treatment or prevention. The invention also includes a method of treating or preventing malaria comprising administering to a patient in need thereof an amount of the (+)-enantiomer of compound (7), or pharmaceutically acceptable salt thereof, free of the (−)-enantiomer of compound (7), or pharmaceutically acceptable salt thereof, sufficient to effect the treatment or prevention, as well as to a method of treating or preventing malaria comprising administering to a patient in need thereof an amount of the (−)-enantiomer of compound (7), or pharmaceutically acceptable salt thereof, free of the (+)-enantiomer of compound (7), or pharmaceutically acceptable salt thereof, sufficient to effect the treatment or prevention. Optimum amounts to be administered and preferred routes of administration can be determined by one skilled in the art. (See also U.S. Pat. No. 6,664,397.)

Again referring to the compounds depicted in FIG. 9, the invention additionally includes a method of treating (or slowing the progression of) symptoms (e.g., dementia) associated with Alzheimer's disease comprising administering to a patient in need thereof an amount of the (+)-enantiomer of compound (14), or pharmaceutically acceptable salt thereof, free of the (−)-enantiomer of compound (14), or pharmaceutically acceptable salt thereof, sufficient to effect the treatment. Alternatively, the invention includes a method of treating (or slowing the progression of) symptoms (e.g., dementia) associated with Alzheimer's disease comprising administering to a patient in need thereof an amount of the (−)-enantiomer of compound (14), or pharmaceutically acceptable salt thereof, free of the (+)-enantiomer of compound (14), or pharmaceutically acceptable salt thereof, sufficient to effect the treatment. Optimum amounts to be administered and preferred routes of administration can be determined by one skilled in the art. (See also U.S. Pat. Nos. 6,372,760, 6,245,911, 6,140,321 and 5,985,864.)

Also referring to the compounds depicted in FIG. 9, the invention further includes a method of treating or preventing nausea or vomiting caused, for example, by radiation therapy or chemotherapy, or surgery, comprising administering to a patient in need thereof an amount of the (+)-enantiomer of compound (17) free of the (−)-enantiomer of compound (17) sufficient to effect the treatment or prevention. Alternatively, the invention includes a method of treating or preventing nausea or vomiting caused, for example, by radiation therapy or chemotherapy, or surgery, comprising administering to a patient in need thereof an amount of the (−)-enantiomer of compound (17) free of the (+)-enantiomer of compound (17) sufficient to effect the treatment. Optimum amounts to be administered and preferred routes of administration can be determined by one skilled in the art. (See also U.S. Pat. Nos. 5,578,628 and 4,753,789.)

Certain aspects of the invention can be described in greater detail in the non-limiting Examples that follows.

### Example 1

Hydrazones possessing an electron withdrawing group (1) (see FIG. 4) termed activated hydrazones, are readily formed from the corresponding substituted hydrazines (e.g., hydrazides, sulfonyl hydrazides, etc.) and ketones and aldehydes under mild conditions, and are rapidly hydrolyzed under similarly mild conditions, making them excellent candidates for auxiliary-based synthetic methods. It was thought that the enhanced acidity of these activated hydrazones would enable rapid metallation over a range of temperatures. Moreover, it was reasoned that the substantial electron density imparted to the electron withdrawing group in the derived azaenolates (2) would lead to tight metal cation binding (see FIG. 4). In an asymmetric context, this could potentially be leveraged to enable high facial selectivity during alkylation, even at temperatures well above −110° C. Collectively, these factors suggested that chiral hydrazines substituted with a conjugative electron withdrawing group could provide the basis of an operationally simple method for asymmetric ketone or aldehyde α-functionalization.

### Experimental Details

General procedure for oxazolidinone N-amination (preparation of 6): n-BuLi (2.5 M in hexanes, 11.4 mL, 28.6 mmol) was added dropwise over ca. 10 min to a stirred and cooled (−78° C.) suspension of 7,7-dimethylnorbornane-(1S,2R)-oxazolidinone (prepared from (+)-camphor sulfonic acid (Yan et al, Tetrahedron Lett. 32:4959-4962 (1991)) (4.32 g, 23.9 mmol) in THF (350 mL) (Ar atmosphere). Ph₂P(O)ONH₂ (6.67 g, 28.6 mmol) was then added and the mixture was removed from the cold bath, stirred for 12 h, filtered and evaporated under reduced pressure to give a yellow solid. Flash chromatography over silica gel using 25:75 EtOAc-hexanes gave 6 (4.4 g, 94%) as a pure, white solid. ¹H NMR (CDCl₃, 400 MHz): δ 4.16 (dd, J=8.2, 4.1 Hz, 1H), 3.91 (s, 2H), 2.30-2.10 (m, 2H), 2.05-1.70 (m, 3H), 1.36-1.24 (m, 1H), 1.18 (s, 3H), 1.0 (s, 3H); ¹³C NMR (CDCl₃, 400 MHz): δ 160.2, 83.2, 72.1, 47.3, 42.7, 35.1, 25.8, 25.4, 20.7, 19.5; ESI-MS m/z [M+H]⁺ calcd for C₁₀H₁₇N₂O₂: 197.26, found 197.1.

General procedure for hydrazone formation (preparation of 11): p-TsOH.H₂O (0.96 g, 5.05 mmol) was added to a stirred solution of 6 (6.144 g, 31.31 mmol) and 3-pentanone (3.95 mL, 37.28 mmol) in CH₂Cl₂ (300 mL) (Ar atmosphere). The mixture was refluxed for 18 h, cooled to rt, and partitioned between CH₂Cl₂ and saturated aqueous NaHCO₃. The organic phase was washed with brine, dried (MgSO₄), filtered and evaporated under reduced pressure to give a yellow oil. Flash chromatography over silica gel using 10:90 EtOAc-hexanes gave 11 (7.645 g, 92%) as a pure, white solid. ¹H NMR (CDCl₃, 400 MHz): δ 4.25 (dd, J=8.2, 4.1 Hz, 1H), 2.50-2.20 (m, 4H), 2.10-1.80 (m, 4H), 1.76 (t, J=4.4 Hz, 1H), 1.32-1.24 (m, 1H), 1.23 (s, 3H), 1.15 (s, 3H), 1.13 (t, J=7.4 Hz, 3H), 1.07 (t, J=7.4 Hz, 3H); ¹³C NMR (CDCl₃, 400 MHz): δ 181.6, 155.3, 82.9, 73.3, 47.9, 42.9, 35.5, 29.1, 26.6, 25.8, 21.4, 19.3, 10.7, 10.5; ESI-MS m/z [M+H]⁺ calcd for C₁₅H₂₅N₂O₂: 265.37, found 265.1.

General procedure for hydrazone alkylation (preparation of 15): n-BuLi (2.5 M in hexanes, 11.65 mL, 29.13 mmol) was added dropwise over ca. 2 min to a stirred and cooled (−78° C.) solution of diisopropylamine (4.45 mL, 31.77 mmol) in THF (0.6 mL) (Ar atmosphere). The mixture was transferred to an ice-H₂O bath, stirred for 30 min, and then cooled to −40° C. A solution of 11 (7.002 g, 26.48 mmol) in THF (260 mL) was added by cannula, with additional THF (2×2.0 mL) as a rinse, and the mixture was stirred for 45 min. Allyl bromide (2.52 mL, 29.13 mmol) was then added and stirring was continued for 5 min. The cold bath was removed and the mixture was stirred for an additional 40 min and then partitioned between Et₂O and H₂O. The aqueous phase was extracted with Et₂O (twice) and the combined organic extracts were washed with brine, dried (MgSO₄), filtered and evaporated under reduced pressure to give a yellow oil. Flash chromatography over silica gel using 10:90 EtOAc-hexanes gave 15 (7.899 g, 98%) as a pure, light-yellow oil. ¹H NMR (CDCl₃, 400 MHz): δ 5.90-5.70 (m, 1H), 5.18-4.94 (m, 2H), 4.25 (dd, J=8.1, 4.1 Hz, 1H), 3.18-3.04 (m, 1H), 2.50-2.24 (m, 4H), 2.14-1.80 (m, 4H), 1.76 (t, J=4.4 Hz, 1H), 1.26-1.32 (m, 2H), 1.23 (s, 3H), 1.16 (s, 3H), 1.13 (t, J=7.2 Hz, 3H), 0.94 (d, J=7.0 Hz, 3H); ¹³C NMR (CDCl₃, 400 MHz): δ 184.4, 155.5, 136.6, 116.7, 82.9, 73.4, 47.9, 43.1, 37.6, 35.6, 35.1, 26.7, 25.8, 24.8, 21.5, 19.3, 17.3, 10.4; ESI-MS m/z [M+H]⁺ calcd for C₁₈H₂₉N₂O₂: 305.44, found 305.1.

General procedure for hydrazone hydrolysis and ACC recovery (preparation of 7 and 6, respectively): p-TsOH.H₂O (9.424 g; 49.54 mmol) was added to a stirred solution of 15 (7.541 g, 24.77 mmol) in acetone (100 mL). The mixture was stirred for 15 min and then partitioned between Et₂O and saturated aqueous NaHCO₃. The aqueous phase was extracted with Et₂O (twice) and the combined organic extracts were washed with brine, dried (MgSO₄), filtered and evaporated under reduced pressure to give a colourless oil that was used directly for chiral GC analysis (performed on a 20 m×0.25 mm Chiraldex GTA column (Advanced Separation Technologies). Analysis was conducted under conditions (50° C.; 15 psi) that gave base-line separation of the enantiomers of an independently prepared racemic mixture of 7. Flash chromatography of the remaining crude reaction mixture over silica gel using 5:95 Et₂O-pentane gave 7 (2.933 g, 94%) as a pure, colorless oil. Spectroscopic data was identical to that reported previously (Hightower et al, J. Org. Chem. 35:1881-1886 (1970)). Continued flash chromatography using 25:75 EtOAc-hexanes gave 38 (5.737 g, 98%) as a pure, white solid. ¹H NMR (CDCl₃, 400 MHz): δ 4.25 (dd, J=8.1, 4.1 Hz, 1H), 3.40-2.26 (m, 1H), 2.08 (s, 3H), 2.06-1.96 (m, 2H), 1.95 (s, 3H), 1.90-1.70 (m, 2H), 1.34-1.24 (m, 1H), 1.23 (s, 3H), 1.20-1.16 (m, 1H), 1.14 (s, 3H); ¹³C NMR (CDCl₃, 400 MHz): δ 173.3, 155.1, 83.1, 73.2, 48.1, 42.9, 35.5, 26.8, 25.8, 25.5, 21.4, 20.1, 19.3; ESI-MS m/z [M+H]⁺ calcd for C₁₃H₂₁N₂O₂: 237.32, found 237.1. 38 (5.729 g; 24.24 mmol) was then combined with HONH₂.HCl (6.731 g; 96.86 mmol) in 4:1 THF—H₂O (250 mL) and stirred for 6 h. The resulting solution was concentrated and partitioned between EtOAc and saturated aqueous NaHCO₃. The aqueous phase was extracted with EtOAc (twice), and the combined organic extracts were washed with brine, dried (MgSO₄), filtered and evaporated to give a light-yellow solid. Flash chromatography over silica gel using 25:75 EtOAc-hexanes gave 6 (4.519 g, 95%) as a pure, white solid.

### Results

Initial studies focused on the easily accessible ACCs (Qin et al, Tet. Lett. 59:393-6402 (2003), Friestad et al, J. Am. Chem. Soc. 122:8329-8330 (2000)). Thus, 3 (see FIG. 4) was prepared by amination of the corresponding oxazolidinone, and was then condensed with 3-pentanone to give 8 (see Table 1). Activated hydrazone 8 was readily deprotonated (LDA, −78° C.) and allylated in excellent (90%) yield. The auxiliary was easily removed and recovered quantitatively, giving 7R/7S in a 76:24 ratio. The analogous sequence with ACC 5 (see FIG. 4) gave better asymmetric induction (86:14). Suspecting that an increase in steric bulk near the amino function was responsible for the increased selectivity, ACC 4 (see FIG. 4) was examined. Indeed, alkylation of the derived hydrazone 10 (see Table 1) gave 7R/7S in a ratio of 91:9. The enantiomeric ratio was further improved to 96:4 using the more conformationally rigid ACC 6 (see FIG. 4 and Table 1).

TABLE 1
Asymmetric Allylation of ACC Hydrazones.

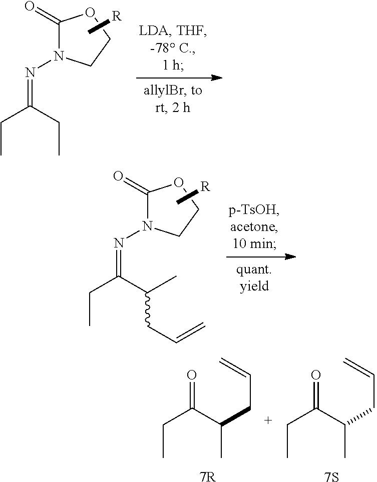

| | | | Allylated | | |
|---|---|---|---|---|---|
| Entry | ACC | Hydrazone | hydrazone | Yield (%) | 7R:7S |
| 1 | 3 | 8 | 12 | 90 | 76:24 |
| 2 | 5 | 9 | 13 | 82 | 86:14 |
| 3 | 4 | 10 | 14 | 93 | 91:9 |
| 4 | 6 | 11 | 15 | 96 | 96:4 |

Allylation via auxiliaries 4 and 6 (see FIG. 4) was studied under a variety of conditions (see Table 2). Of the bases evaluated, use of LDA gave the highest stereoselectivity and showed no solvent dependence. Asymmetric induction proved largely independent of temperature; the same high level of selectivity was obtained when the alkylation was conducted up to −40° C., with only a slight decrease at temperatures up to 0° C.

TABLE 2
Effect of Reaction Conditions on Stereoselectivity.

| Entry | Hydrazone | Base | Solvent | Temp (° C.) | 7R:7S |
|---|---|---|---|---|---|
| 1 | 11 | LDA | THF | −78 to rt | 96:4 |
| 2 | 11 | LDA | Et₂O | −78 to rt | 96:4 |
| 3 | 11 | LDA | toluene | −78 to rt | 96:4 |
| 4 | 11 | LHMDS | THF | −78 to rt | 87:13 |
| 5 | 11 | NHMDS | THF | −78 to rt | 82:18 |
| 6 | 11 | KHMDS | THF | −78 to rt | 82:18 |
| 7 | 11 | LDA | THF | −110 to rt | 96:4 |
| 8 | 11 | LDA | THF | −60 to rt | 96:4 |
| 9 | 11 | LDA | THF | −40 to rt | 96:4 |
| 10 | 11 | LDA | THF | −20 to rt | 91:9 |
| 11 | 11 | LDA | THF | 0 to rt | 90:10 |
| 12 | 10 | LDA | THF | −110 to rt | 91:9 |
| 13 | 10 | LDA | THF | −78 to rt | 91:9 |
| 14 | 10 | LDA | THF | −60 to rt | 90:10 |
| 15 | 10 | LDA | THF | −40 to rt | 91:9 |
| 16 | 10 | LDA | THF | −20 to rt | 85:15 |
| 17 | 10 | LDA | THF | 0 to rt | 86:14 |

The scope of the reaction was examined with ACC 4 and 6 (see FIG. 4 and Table 3). Excellent yield and stereoselectivity resulted for each alkyl halide examined, including a 2° alkyl iodide (entry 6). ACC 6 consistently outperformed 4 in terms of asymmetric induction, and gave results comparable with literature reports yet with considerably improved isolated yields (for example, alkylation via SAMP hydrazones gives: 31 (61%; β:α=97.3), 32 (60%; β:α>99:1), 37 (60%; (β:α=86:14) (Enders In Asymmetric Synthesis, 1^{st} ed; (Ed.: J. D. Morrison) Academic Press: New York 3:275-339 (1984))), Notably, alkylation via ACCs is also very easy to carry out: hydrazone formation and cleavage are straightforward and efficient, with no damage or loss of the auxiliary, and the azaenolate is readily formed and alkylated at temperatures up to 0° C. The simplicity and mildness of this method gives rise to the possibility of convenient large-scale asymmetric α-alkylation of ketones. As a preliminary test of this, the allylation was carried out using 7.002 g of 11, which was greater than a 100 fold increase over the initial experiments. Exposure of 11 to LDA for 45 min at −40° C., followed by addition of allyl bromide and stirring for 45 min, gave 15 in 98% yield. Hydrolysis with p-TsOH.H₂O in acetone (15 min) gave ketone 7 in 94% yield with an unchanged enantiomeric ratio (96:4), along with acetone-derived hydrazone 38 in 98% yield. Treatment of 38 with HONH₂.HCl in THF—H₂O gave the recovered ACC auxiliary (6) in 95% yield.

TABLE 3
Asymmetric Alkylation via ACC 4 and 6.

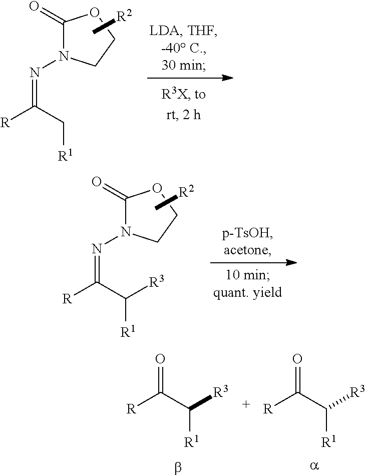

| | | | | | | Alkylated | Yield | | |
|---|---|---|---|---|---|---|---|---|---|
| Entry | R | R¹ | ACC | Hydrazone | R³X | hydrazone | (%) | Ketone | β:α |
| 1 | Et | Me | 6 | 11 | allylBr | 15 | 96 | 7 | 96:4 |
| 2 | Et | Me | 6 | 11 | BnBr | 19 | 99 | 30 | 96:4 |
| 3 | Et | Me | 6 | 11 | EtI | 20 | 92 | 31 | 97:3 |
| 4 | Et | Me | 6 | 11 | PrI | 21 | 89 | 32 | 96:4 |
| 5 | Et | Me | 6 | 11 | PrOTs | 21 | 76 | 32 | 85:15 |
| 6 | Et | Me | 6 | 11 | i-PrI | 22 | 77 | 33 | 94:6 |
| 7 | Et | Me | 6 | 11 | ArCH₂Br^{a} | 23 | 93 | 34 | 96:4 |
| 8 | Ph | Me | 6 | 16 | allylBr | 24 | 91 | 35 | 96:4 |
| 9 | i-Pr | Me | 6 | 17 | allylBr | 25 | 88 | 36 | 98:2 |
| 10 | —(CH₂)₄— | | 6 | 18 | allylBr | 26 | 91 | 37 | 82:18 |
| 11 | Et | Me | 4 | 10 | allylBr | 14 | 93 | 7 | 91:9 |
| 12 | Et | Me | 4 | 10 | BnBr | 27 | 98 | 30 | 92:8 |
| 13 | Et | Me | 4 | 10 | EtI | 28 | 83 | 31 | 90:10 |
| 14 | Et | Me | 4 | 10 | PrI | 29 | 77 | 32 | 92:8 |

^{a}Ar = 4-Br—C₆H₄

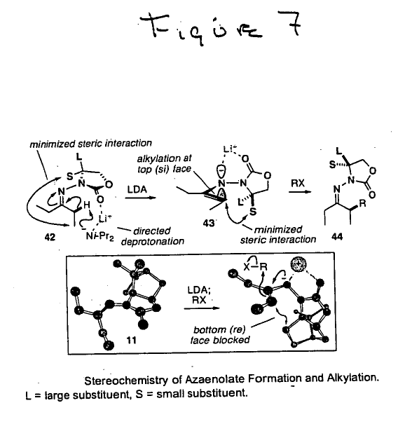

Crystal structures of the major diastereomer of 23 and 26 (see FIG. 6) were obtained and showed that alkylation occurs syn to the auxiliary, relative to the CN double bond, indicating that the azaenolate intermediate likely has the Z-geometry (Z_{CN}) about this bond. Furthermore, alkylation in each case (11→23; 18→26) (see Table 3) provided the same sense of chirality at the newly-formed stereogenic center, implying that, like cyclic compound 18, the acyclic systems react via the azaenolate having the E-configuration (E_{CC}) at the carbon-carbon bond.

The regioselectivity of the alkylation was consistent with a directing effect occurring during deprotonation, which could provide a convenient and general means of overriding the inherent selectivity of LDA. Moreover, in an asymmetric context, this would make the direct synthesis of optically enriched α,α-disubstituted ketones possible for the first time. To test this idea, 38 was subjected to allylation giving 39 regioselectively in 94% yield as a single double-bond diastereomer (FIG. 5). Alkylation of 39 also proceeded regioselectively to give the α,α- and the α,α′-bisalkylation products 41 (97:3 diastereomeric ratio; major shown) and 40, respectively, in a 92:8 ratio, thus demonstrating the concept of directed deprotonation (FIG. 5). In contrast LDA-mediated bisalkylation of ketones (d'Andelo, Tetrahedron 32:2979-2990 (1976)), imines (Meyers, J. Am. Chem. Soc. 98:3032-3033 (1976), Meyers et al, J. Am. Chem. Soc. 103:3081-3087 (1981), Hashimoto et al, Tet. Lett. 573-576 (1978), Hashimoto et al, Chem. Pharm. Bull. 27:2760-2766 (1979)) and dialkyl hydrazones (Enders in Assymetric Synthesis, 1^{st} ed, (Ed. J. D. Morrison), Academic Press, New York 3:275-339 (1984); Job et al, Tetrahedron 58:2253-2329 (2002)) gives α,α′-bisalkylation. This appears to be the first instance of not only directed deprotonation in azaenolate formation via a neutral coordinating element (Adlington et al, Acc. Chem. Res. 16:55-59 (1983), Kofron et al, J. Org. Chem. 41:439-442 (1976)), but also asymmetric α,α-bisalkylation of a ketone.

A stereochemical model consistent with the above observations is shown in FIG. 7. Deprotonation of 42 gives azaenolate 43 that is then alkylated from its less-hindered face to form 44. The E_{cc} geometry of 43 originates from minimization of steric interactions between the syn β-methyl group and the auxiliary in 42, and directed deprotonation via coordination of the carbonyl oxygen and LDA sets the Z_{CN} configuration. In this form, the bottom (re) face of the azaenolate is blocked, causing the electrophile to approach from the top (si) face.

In summary, described above is a convenient and practical method for asymmetric α-alkylation and α,α-bisalkylation of ketones via ACC chiral auxiliaries (FIG. 8). In contrast to other methods, the auxiliaries are both easily introduced into and removed from ketones, with quantitative recovery. Furthermore, deprotonation is rapid, and alkylation does not require extreme low temperature, yet proceeds with excellent stereoselectivity and substantially greater yields. Collectively, these traits render the prospect of large-scale asymmetric ketone α-alkylation, which has previously not been possible. Furthermore, the ACC auxiliaries exhibit a unique directing effect that overrides the inherent selectivity of LDA enabling, for the first time, the asymmetric α,α-bisalkylation of ketones. Further study of this directing effect, and the mechanistic details, scope and synthetic utility of this reaction are underway.

### Example 2

A single enantiomer form of the antimalarial mefloquine (LARIAM® (compound (11) in FIG. 9 when R is H and present as the hydrochloride salt)), or equivalents of mefloquine, can be prepared by one of either two methods (FIG. 9A). In the first method, the activated hydrazone of cyclopentanone (compound (2)), prepared in a manner as described above, is deprotonated and then reacted with 2,8-bis(trifluoromethyl)-4-quinolinecarboxaldehyde, or its equivalent. The activated hydrazone (compound (3)) is then hydrolyzed and transformed into its oxime (compound (5)). Through a Beckmann rearrangement, the oxime is converted to the amide (compound 6)), which is then deoxygenated to yield compound (7). The second method begins with the activated hydrazone of 3-piperidone (compound (9)), or its equivalent, being deprotonated and then allowed to react with 2,8-bis(trifluoromethyl)-4-quinolinecarboxaldehyde, or its equivalent. Hydrolysis of the activated hydrazone (compound (10) followed by deoxygenation gives mefloquine (compound (11)) or its equivalent stereoisomers.

A single enantiomer form of the cholinesterase inhibitor donepezil (ARICEPT® (compound (14) in FIG. 9B when present as the hydrochloride salt), or equivalents of donepezil, can be prepared from the activated hydrazone of 3,4-dimethoxyindanone (compound (13)) or its equivalent (FIG. 9B). The activated hydrazone is deprotonated and then alkylated with 1-benzyl-4-(bromomethyl)piperidine or an equivalent alkylating reagent. Preparation is completed by the hydrolysis of the activated hydrazone to give compound (14).

A single enantiomer form of the serotonin blocker ondanestron (ZOFRAN® (compound (17) in FIG. 9C), or equivalents of ondanestron, can be prepared from the activated hydrazone of 9-methyl-2,3-dihydro-1H-carbazol-4(9H)-one (compound (15)) or other ketone equivalents (FIG. 9C). The activated hydrazone (compound (16)) is deprotonated and then alkylated with 1-(bromomethyl)-2-methyl-1H-imidazole or equivalent alkylating reagents. Preparation is completed by the hydrolysis of the activated hydrazone to give compound (17).

All documents and other information sources cited above are hereby incorporated in their entirety by reference.

What is claimed is:

1. A process for effecting asymmetric α-functionalization of a compound of Formula I 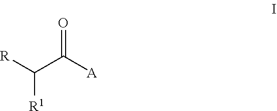
comprising:
i) reacting the compound of Formula I with a substituted hydrazide of the formula H₂N—C (auxiliary) under conditions such that an activated hydrazone of Formula II is formed 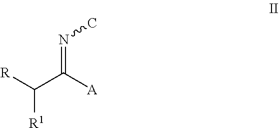
ii) reacting said activated hydrazone of Formula II with a base capable of deprotonating said activated hydrazone and with an electrophile (E) under conditions such that a substituted activated hydrazone of Formula III or IV is produced 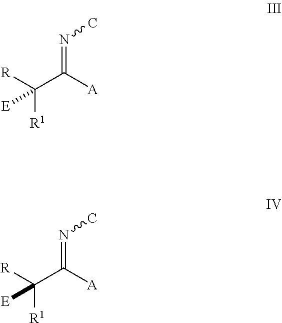
iii) hydrolyzing said substituted activated hydrazone of Formula III or IV to yield said asymmetrically functionalized compound and said auxiliary,
wherein
A=H or alkyl or substituted alkyl, 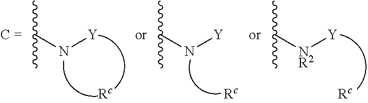
Y=electron withdrawing group,
R^{c}=chiral group,
R, R¹ and R² are, independently, alkyl (linear, branched or cyclic), substituted alkyl (linear, branched or cyclic), aryl, substituted aryl, heteroatom, halogen, Si, or B.

2. The process according to claim 1 wherein said α-functionalization is α-alkylation, amination, halogenation, oxidation or thiolation.

3. The method according to claim 1 wherein said auxiliary is a chiral N-amino cyclic carbamate or a chiral N-amino cyclic sulfamate.

4. A process for effecting asymmetric α,α-bisfunctionalization of a compound of Formula I′ 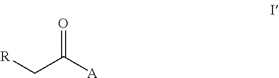
comprising:
i) reacting the compound of Formula I′ with a substituted hydrazide of formula H₂N—C (auxiliary) under conditions such that an activated hydrazone of Formula II′ is formed 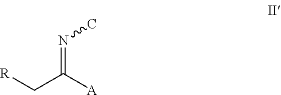
ii) reacting said activated hydrazone of Formula II with a base capable of deprotonating said activated hydrazone and with a first electrophile (E) under conditions such that a substituted activated hydrazone of Formula III′ or IV′ is produced 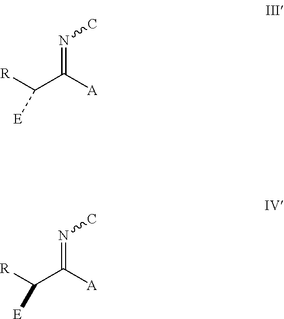
iii) reacting said substituted activated hydrazone of Formula III′ or IV′ with a base capable of deprotonating said substituted activated hydrazone of Formula III′ or IV′ and with a second electrophile (E′) under conditions such that a disubstituted activated hydrazone of Formula V or VI is produced 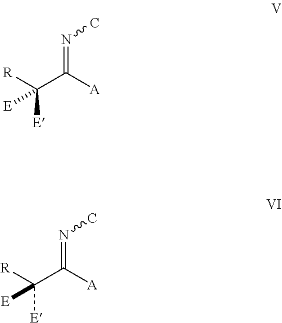
iv) hydrolyzing said disubstituted activated hydrazone of Formula V or VI to yield said asymmetrically bisfunctionalized compound and said auxiliary,
wherein
A=H or alkyl or substituted alkyl, 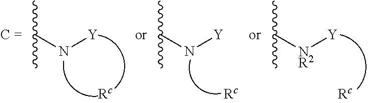
Y=electron withdrawing group,
R^{c}=chiral group,
R and R² are, independently, alkyl (linear, branched or cyclic), substituted alkyl (linear, branched or cyclic), aryl, substituted aryl, heteroatom, halogen, Si, or B.

5. A process for effecting asymmetric α-functionalization of a compound of Formula I 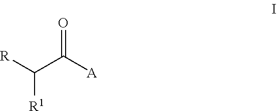
comprising:
i) reacting the compound of Formula I with a substituted hydrazide of the formula H₂N—C (auxiliary) under conditions such that an activated hydrazone of Formula II is formed 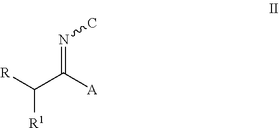
ii) reacting said activated hydrazone of Formula II with a base capable of deprotonating said activated hydrazone and with an electrophile (E) under conditions such that a substituted activated hydrazone of Formula III or IV is produced 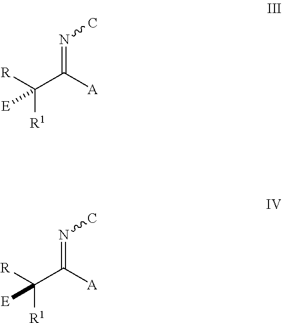
iii) hydrolyzing said substituted activated hydrazone of Formula III or IV to yield said asymmetrically functionalized compound and said auxiliary,
wherein
A=H or alkyl (linear, branched or cyclic) or substituted alkyl (linear, branched or cyclic), 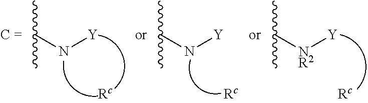
Y=electron withdrawing group,
R^{c}=chiral group, and
R, R¹ and R² are, independently, H, alkyl (linear, branched or cyclic), substituted alkyl (linear, branched or cyclic), aryl, substituted aryl, heteroatom, halogen, Si, or B, or
A, R and R¹, A and R or A and R¹ together are a cyclic alkyl or a heterocycle.

6. A compound of the formula: 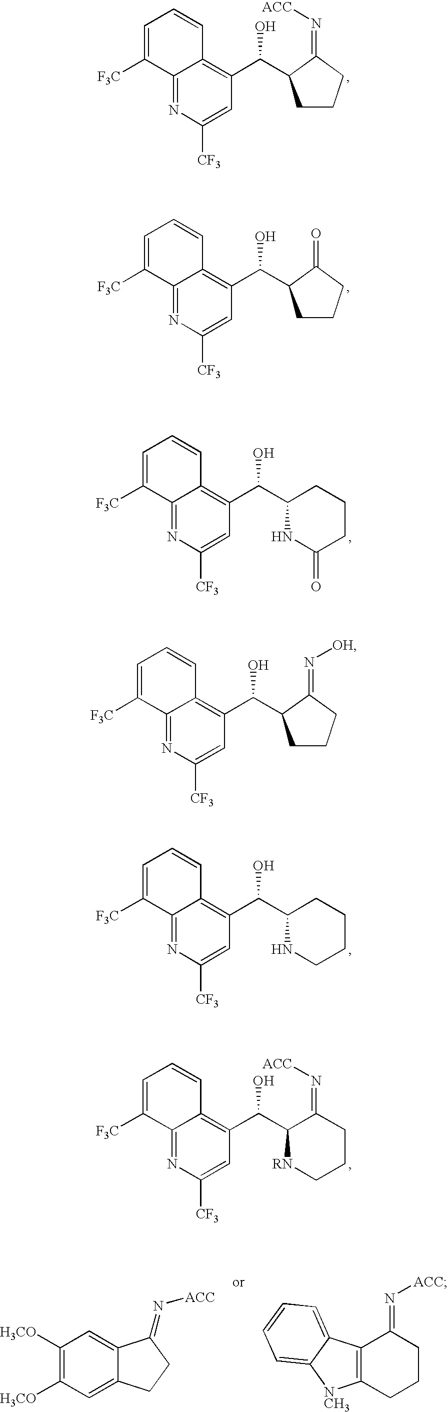
or pharmaceutically acceptable salt thereof.

7. The (+)-enantiomer of a compound of formula: 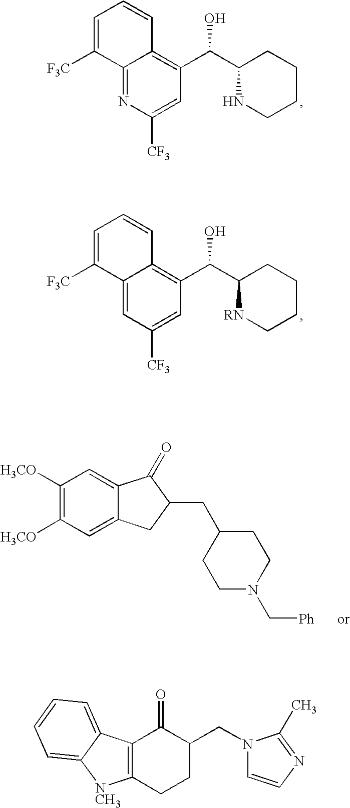
or pharmaceutically acceptable salt thereof, free of the (−)-enantiomer of said compound, or pharmaceutically acceptable salt thereof.

8. The (−)-enantiomer of a compound of formula: 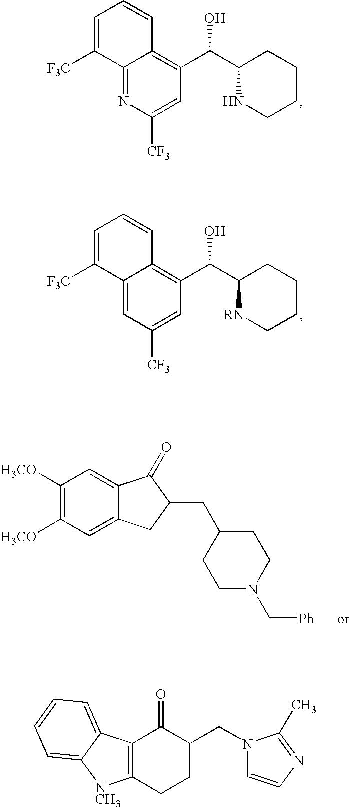
or pharmaceutically acceptable salt thereof, free of the (+)-enantiomer of said compound, or pharmaceutically acceptable salt thereof.

9. A composition comprising the (+)-enantiomer according to claim 7 and a pharmaceutically acceptable carrier.

10. A composition comprising the (−)-enantiomer according to claim 7 and a pharmaceutically acceptable carrier.

11. A method of treating or preventing malaria comprising administering to a patient in need thereof an amount of a single enantiomer of compound (7) or compound (11) of FIG. 9A, or pharmaceutically acceptable salt thereof, sufficient to effect the treatment or prevention.

12. A method of treating, or slowing the progression of, symptoms associated with Alzheimer's disease comprising administering to a patient in need thereof an amount of a single enantiomer of compound (14) of FIG. 9B, or pharmaceutically acceptable salt thereof, sufficient to effect said treatment or slow said progression.

13. A method of treating or preventing nausea or vomiting comprising administering to a patient in need thereof an amount of a single enantiomer of compound (17) of FIG. 9C, or pharmaceutically acceptable salt thereof, sufficient to effect said treatment or prevention.
